(19) United States
(12) Patent Application Publication
(10) Pub. No.: US 2024/0032896 A1
(43) Pub. Date: Feb. 1, 2024

(54) ULTRASONIC DIAGNOSIS APPARATUS, MEDICAL IMAGE PROCESSING APPARATUS, AND NON-TRANSITORY COMPUTER READABLE MEDIUM

(71) Applicant: CANON MEDICAL SYSTEMS CORPORATION, Otawara-shi (JP)

(72) Inventors: Masashi USUMURA, Nasushiobara (JP); Koichiro KURITA, Nasushiobara (JP); Itsuki KUGA, Utsunomiya (JP)

(73) Assignee: CANON MEDICAL SYSTEMS CORPORATION, Otawara-shi (JP)

(21) Appl. No.: 18/359,326

(22) Filed: Jul. 26, 2023

(30) Foreign Application Priority Data
Jul. 27, 2022 (JP) 2022-119658

(51) Int. Cl. A61B 8/08; A61B 8/00

(52) U.S. Cl. CPC A61B 8/5246; A61B 8/488; A61B 8/465

(57) ABSTRACT

An ultrasonic diagnosis apparatus according to an embodiment includes a processor. The processor acquires Doppler data that is obtained through ultrasonic transmission/reception in a Doppler mode and information about a type of the Doppler mode. The processor determines at least one executable measurement item for the Doppler data based on the information about the type of the Doppler mode.

### CROSS-REFERENCE TO RELATED APPLICATION(S)

This application is based upon and claims the benefit of priority from Japanese Patent Application No. 2022-119658, filed on Jul. 27, 2022; the entire contents of which are incorporated herein by reference.

### BACKGROUND OF THE INVENTION

### 1. Field of the Invention

Embodiments disclosed in the present specification and drawings relate to an ultrasonic diagnosis apparatus, a medical image processing apparatus, and a non-transitory computer readable medium.

### 2. Description of the Related Art

Ultrasonic diagnosis apparatuses are conventionally diagnosis apparatuses for imaging information about inside of a living organism and providing it, and various measurements are performed with them. Some ultrasonic diagnosis apparatuses are applied to, for example, diagnosis related to the circulatory system, etc. A user, such as an engineer, acquires Doppler data through ultrasonic transmission/reception in a Doppler mode by an ultrasonic apparatus and performs measurement for various items.

With measurement based on a Doppler mode, however, there exist two or more workflows for measurement, and there exist in the workflows many measurement items possible to become a target of measurement; therefore, the user needs a time to decide which item is to be measured, and thus further improvement is required.

### BRIEF DESCRIPTION OF THE DRAWINGS

FIG. 1 is a block diagram illustrating an example of structure of an ultrasonic diagnosis apparatus according to an embodiment;

FIG. 2 is a diagram illustrating an exemplary ultrasonic image;

FIG. 3 is a schematic diagram illustrating exemplary measurement items of the ultrasonic diagnosis apparatus according to the embodiment; and

FIG. 4 is a flowchart illustrating an example of processing procedure performed by the ultrasonic diagnosis apparatus according to the embodiment.

### DETAILED DESCRIPTION OF THE PREFERRED EMBODIMENTS

An ultrasonic diagnosis apparatus according to an embodiment includes a processor. The processor acquires Doppler data that is obtained through ultrasonic transmission/reception in a Doppler mode and information about a type of the Doppler mode. The processor determines at least one executable measurement item for the Doppler data based on the information about the type of the Doppler mode.

Described in detailed below are an ultrasonic diagnosis apparatus, a medical image processing apparatus, and a non-transitory computer readable medium according to the present application with reference to the accompanied drawings. Note that the present embodiment targets the circulatory system of a subject and, for explanation easy to understand, performs measurement for a left-ventricle diastolic dysfunction, especially. Note that an ultrasonic diagnosis apparatus, a medical image processing apparatus, and a non-transitory computer readable medium according to the present application are not limited by embodiments as described below. In the explanation below, the same reference numerals/signs are denoted with the same components, and redundant explanation is not made.

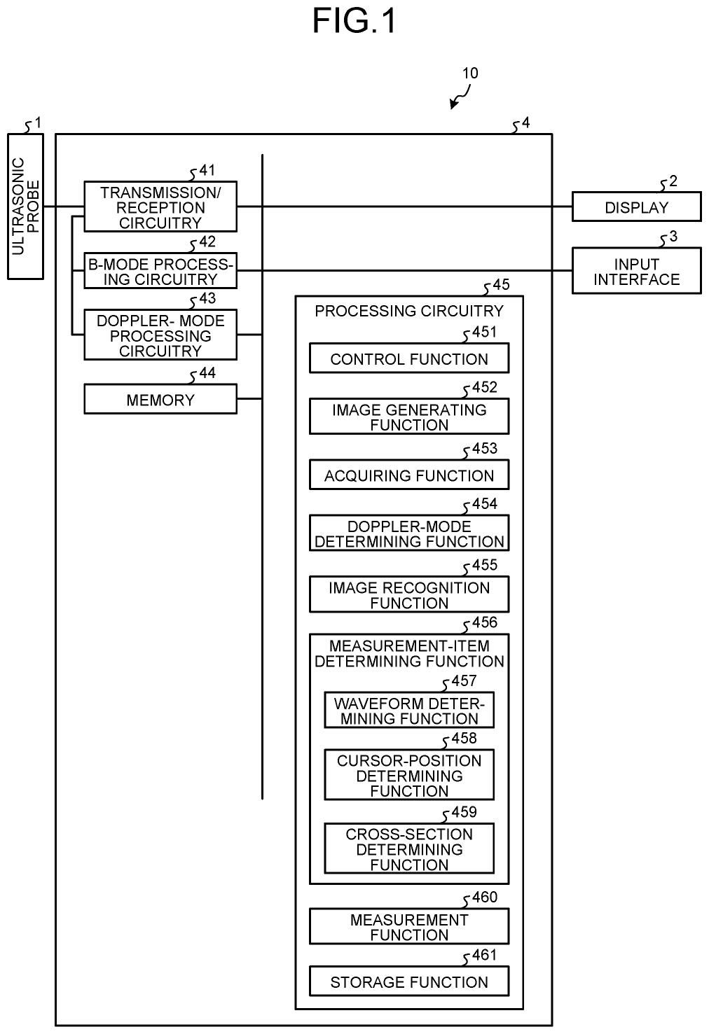

FIG. 1 is a block diagram illustrating an example of structure of an ultrasonic diagnosis apparatus 10 according to a first embodiment. As illustrated in FIG. 1, the ultrasonic diagnosis apparatus 10 according to the present embodiment includes an ultrasonic probe 1, a display 2, an input interface 3, and a body 4, in which the ultrasonic probe 1, the display 2, and the input interface 3 are connected to the body 4 such that they can communicate with the body 4. The ultrasonic diagnosis apparatus 10 is an example of an ultrasonic diagnosis apparatus and a medical image processing apparatus.

The ultrasonic probe 1 is connected to transmission/reception circuitry 41 included in the body 4. The ultrasonic probe 1 is an example of an ultrasonic apparatus. The ultrasonic probe 1 has a plurality of piezoelectric vibrators in a probe body, for example. These piezoelectric vibrators generate ultrasonic waves based on a driving signal supplied from the transmission/reception circuitry 41. In addition, the ultrasonic probe 1 receives a wave reflected from the subject and converts the reflected wave into an electric signal.

The ultrasonic probe 1 further has a matching layer provided on the piezoelectric vibrators, a backing material for preventing the ultrasonic wave from transmitting backward from the piezoelectric vibrators, etc. Note that the ultrasonic probe 1 is connected to the body 4 in a detachable manner. The ultrasonic probe 1 is a sector, linear, or convex ultrasonic probe, for example.

The ultrasonic probe 1 transmits a signal that is obtained through ultrasonic transmission/reception to the body 4. More particularly, when ultrasonic waves are transmitted from the ultrasonic probe 1 to the subject, the transmitted ultrasonic waves are reflected one after another by a surface of body tissues of the subject having discontinuous acoustic impedance, and the reflected ultrasonic waves are received by the plurality of piezoelectric vibrators included in the ultrasonic probe 1 as reflected-wave signals.

The amplitude of the reflected-wave signal received depends on a difference between acoustic impedances of a discontinuous surface by which the ultrasonic wave has been reflected. Note that if the transmitted ultrasonic pulse has been reflected by a surface of a moving body, such as a blood flow or a cardiac wall, the reflected-wave signal is subjected to, due to Doppler effects, a frequency shift that depends on a velocity component in an ultrasonic-wave transmitting direction of the moving body.

Note that the present embodiment is applicable either when the subject is scanned two-dimensionally with the ultrasonic probe 1 that is a one-dimensional ultrasonic probe having a plurality of piezoelectric vibrators aligned in a row, or when the subject is scanned three-dimensionally with the ultrasonic probe 1 that mechanically swings a plurality of piezoelectric vibrators of a one-dimensional ultrasonic prove or with the ultrasonic probe 1 that is a two-dimensional ultrasonic probe having a plurality of piezoelectric vibrators arranged two-dimensionally in a grid.

The display 2 displays a graphical user interface (GUI) that is used by the operator of the ultrasonic diagnosis apparatus 10 to input various setting requests with the input interface 3, an ultrasonic image generated by the body 4, etc. Moreover, the display 2 displays various messages or display information to notify the operator of processing state of the body 4, processing results, etc. Furthermore, the display 2 can have a speaker and output sounds.

The input interface 3 is manipulated so that a predetermined position (for example, various positions for measurement) is set, for example. The input interface 3 is implemented by, for example, a trackball, a switch button, a mouse, a keyboard, a touch pad with which an input operation is made through a touch on an operation screen, a touch monitor in which a display screen and a touch pad are integrated together, non-contact input circuitry using an optical sensor, voice input circuitry, and the like. The input interface 3 is connected to later-described processing circuitry 45, and it converts an input operation received from the operator into an electric signal and outputs the electric signal to the processing circuitry 45.

Note that, in the present specification, the input interface 3 is not limited to ones provided with a physical operational member, such as a mouse or a keyboard. Examples of the input interface include, for example, electric-signal processing circuitry that receives an electric signal corresponding to an input operation from an external input device that is provided separately from the ultrasonic diagnosis apparatus 10 and outputs the electric signal to the processing circuitry 45.

The body 4 includes the transmission/reception circuitry 41, B-mode processing circuitry 42, Doppler-mode processing circuitry 43, a memory 44, and the processing circuitry 45. In the ultrasonic diagnosis apparatus 10 as illustrated in FIG. 1, various processing functions are stored in the memory 44 in format of programs executable by a computer.

The transmission/reception circuitry 41, the B-mode processing circuitry 42, the Doppler-mode processing circuitry 43, and the processing circuitry 45 are processors that implement functions corresponding to the respective programs by reading the programs from the memory 44 and executing the programs. In other words, the circuitry is given a function corresponding to a program when it reads the program.

The transmission/reception circuitry 41 receives a signal that has been obtained through ultrasonic transmission/reception by the ultrasonic probe 1. The transmission/reception circuitry 41 includes a pulse generator, transmission delay circuitry, a pulsar, etc., and it supplies a driving signal to the ultrasonic probe 1. The pulse generator repeatedly generates a rate pulse for forming a transmission ultrasonic wave at a predetermined rate frequency. The transmission delay circuitry causes the ultrasonic waves generated from the ultrasonic probe 1 focused to shape as a beam, and it provides each rate pulse generated by the pulse generator with a delay time for each piezoelectric vibrator that is necessary to decide transmission directional characteristics.

The pulsar applies a driving signal (driving pulse) to the ultrasonic probe 1 at timing based on the rate pulse. In other words, the transmission delay circuitry adjusts the transmission directions of the ultrasonic waves transmitted from the piezoelectric vibrator surface as appropriate by changing the delay time provided for each rate pulse.

Note that the transmission/reception circuitry 41 has a function able to change transmission frequency, transmission driving voltage, etc., at once to perform a predetermined scanning sequence based on an instruction of the processing circuitry 45, as described later. Among them, a change in transmission driving voltage is implemented by a linear-amplifier oscillator that is able to switch values at once or a mechanism for electrically switching two or more power supply units.

Moreover, the transmission/reception circuitry 41 includes a pre-amplifier, an analog/digital (A/D) convertor, reception delay circuitry, an adder, etc., and it generates reflected-wave data by performing various processes on the reflected-wave signal received by the ultrasonic probe 1. The pre-amplifier amplifies the reflected-wave signal for each channel. The A/D converters performs A/D conversion on the amplified reflected-wave signal.

The reception delay circuitry provides a delay time that is necessary to decide reception directional characteristics. The adder generates the reflected-wave data by performing an addition process of the reflected-wave signal that has been processed by the reception delay circuitry. The addition process by the adder emphasizes a reflective component from a direction corresponding to the reception directional characteristics of the reflected-wave signal, so that a general beam for ultrasonic transmission/reception is formed by the reception and transmission directional characteristics.

The B-mode processing circuitry 42 receives the reflected-wave data from the transmission/reception circuitry 41, and it performs logarithm amplification, envelope detection processing, etc., thereby generating data (B-mode data) in which signal intensities are represented by levels of brightness.

The Doppler-mode processing circuitry 43 performs frequency analysis on velocity information from the reflected-wave data that is received from the transmission/reception circuitry 41, extracts components of blood flow, tissues, and contrast echo due to the Doppler effects, and generates data (Doppler data) in which information about the moving body, such as velocity, dispersion, power, etc., is extracted for multiple points. The moving body is, for example, a fluid including blood flowing through a blood vessel, lymph flowing through a lymph vessel, or the like.

Note that the B-mode processing circuitry 42 and the Doppler-mode processing circuitry 43 can process both two-dimensional reflected-wave data and three-dimensional reflected-wave data. In other words, the B-mode processing circuitry 42 generates two-dimensional B-mode data from two-dimensional reflected-wave data and generates three-dimensional B-mode data from three-dimensional reflected-wave data. Moreover, the Doppler-mode processing circuitry 43 generates two-dimensional Doppler data from two-dimensional reflected-wave and generates three-dimensional Doppler data from three-dimensional reflected-wave.

Moreover, the B-mode processing circuitry 42 can also generate three-dimensional reflected-wave data by combining two or more sets of two-dimensional reflected-wave data together and generate three-dimensional B-mode data from the generated three-dimensional reflected-wave data. Furthermore, the Doppler-mode processing circuitry 43 can also generate three-dimensional reflected-wave data by combining two or more sets of two-dimensional reflected-wave data together and generate three-dimensional Doppler data from the generated three-dimensional reflected-wave data.

The memory 44 stores therein an ultrasonic image that is generated by the processing circuitry 45 for display. Moreover, the memory 44 can store therein B-mode data generated by the B-mode processing circuitry 42 and Doppler data generated by the Doppler-mode processing circuitry 43. Furthermore, the memory 44 stores therein various data, such as control programs for performing ultrasonic transmission/reception, image processing, and display processing, diagnosis information (for example, patient ID, doctor's finding, etc.), diagnosis protocols, various body marks, etc.

Moreover, the memory 44 can also store therein a trained model that is generated by using, as teacher data, B-mode images labelled with cross-sections of the circulatory system. Furthermore, the memory 44 can also store therein a trained model that is generated by using, as teacher data, Doppler images labelled with selectable measurement items. Note that the trained models are generated by a well-known method.

The processing circuitry 45 controls general processing of the ultrasonic diagnosis apparatus 10. More particularly, the processing circuitry 45 reads programs from the memory 44 and executes the programs to perform various processes, the programs corresponding to a control function 451, an image generating function 452, an acquiring function 453, a Doppler-mode determining function 454, an image recognition function 455, a measurement-item determining function 456, a measurement function 460, and a storage function 461 as illustrated in FIG. 1,

The processing circuitry 45 is a processor that implement a function corresponding to a program by reading the program from the memory 44 and executes the program. In other words, the processing circuitry 45 is given the respective functions as illustrated in FIG. 1 inside the processing circuitry 45, when it reads the respective programs.

The control function 451 is herein an example of a control unit. The image generating function 452 is an example of an image generating unit. The acquiring function 453 is an example of an acquiring unit.

Note that it is explained in the present embodiment that various processing functions as described below are implemented by the single processing circuitry 45. However, it is allowable to form processing circuitry by combining two or more independent processors together such that the functions are implemented by the respective processors executing the programs.

The control function 451 controls processes of the transmission/reception circuitry 41, the B-mode processing circuitry 42, and the Doppler-mode processing circuitry 43 based on various setting requests received from the operator through the input interface 3 and various control programs and various data read from the memory 44. Moreover, the control function 451 performs control such that an ultrasonic image and various information are displayed on the display 2. Furthermore, the control function 451 performs various measurement processes.

The image generating function 452 generates an ultrasonic image from data generated by the B-mode processing circuitry 42 and the Doppler-mode processing circuitry 43. In other words, the image generating function 452 generates an ultrasonic image (hereinafter, referred to as “B-mode image”) from two-dimensional B-mode data generated by the B-mode processing circuitry 42, the B-mode image representing intensities of reflected waves with brightness.

Moreover, the image generating function 452 generates an ultrasonic image (hereinafter, referred to as “Doppler image”) from two-dimensional Doppler data generated by the Doppler-mode processing circuitry 43, the Doppler image representing information about the moving body. The ultrasonic image based on the Doppler data is image data that is velocity image data, dispersion image data, or power image data or a combination thereof, and it includes flow velocity waveform that is obtained by representing the velocity of blood flow at the position of the Doppler cursor as a waveform.

In general, the image generating function 452 herein generates an ultrasonic image for display by converting (scanning-converts) a scanning-line signal train of ultrasonic scanning into a scanning-line signal train for a video format which is represented by a television, etc. More particularly, the image generating function 452 generates an ultrasonic image for display by performing coordinate conversion depending on the scanning format of the ultrasonic wave by the ultrasonic probe 1.

Moreover, the image generating function 452 performs, for example, an image process (smoothing process) for re-generating an image of average brightness by using two or more scanning-converted image frames, an image process (edge enhancement process) in which a differential filter is used inside an image, etc., as various image processes other than the scanning conversion. Furthermore, the image generating function 452 combines text information, scale, body mark, etc., of various parameters with the ultrasonic image.

Moreover, the image generating function 452 generates a three-dimensional ultrasonic image (hereinafter, referred to as “B-mode image”) by performing coordinate conversion for three-dimensional B-mode data generated by the B-mode processing circuitry 42. Furthermore, the image generating function 452 generates a three-dimensional ultrasonic image (hereinafter, referred to as “Doppler image”) by performing coordinate conversion for three-dimensional Doppler data generated by the Doppler-mode processing circuitry 43.

In addition, in order to generate various two-dimensional images so that the three-dimensional image data (volume data) are displayed on the display 2, the image generating function 452 can perform a rendering process for the volume data.

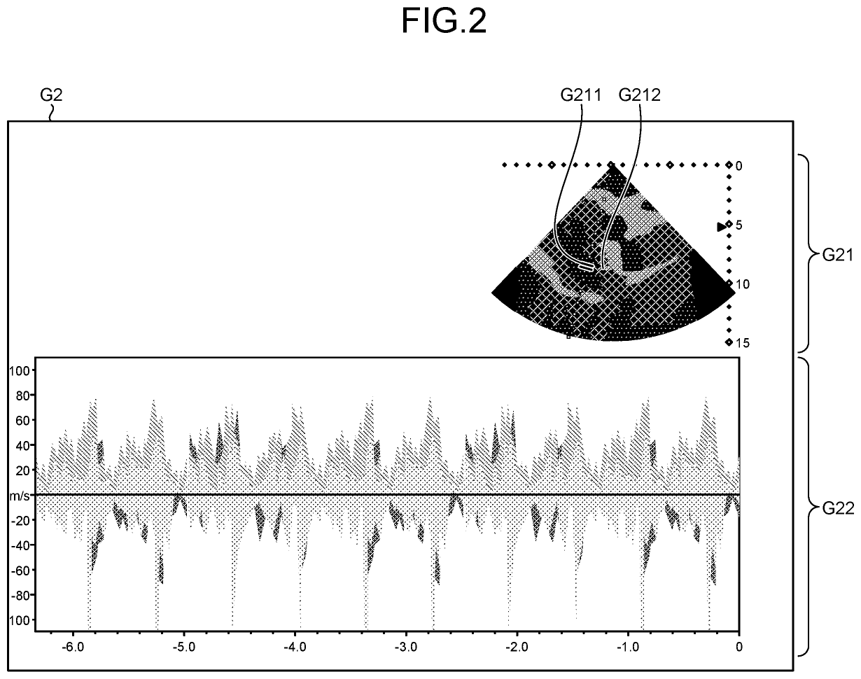

Explained below is an ultrasonic image generated by the image generating function 452 with reference to FIG. 2. FIG. 2 is a diagram illustrating an exemplary ultrasonic image. An ultrasonic image G2 includes a Doppler-diagnosis partial image display region G21 and a Doppler-data display region G22. The control function 451 displays the B-mode image generated by the image generating function 452 on the Doppler-diagnosis partial image display region G21.

The Doppler-diagnosis partial image includes a Doppler cursor image G211 representing a Doppler cursor and an observation object image G212 representing an observation object. The control function 451 displays the Doppler image generated by the image generating function 452 on the Doppler-data display region G22.

Returning back to FIG. 1, the acquiring function 453 acquires the Doppler data that is obtained through ultrasonic transmission/reception in a Doppler mode and information about a type of Doppler mode. More particularly, the acquiring function 453 acquires the Doppler data generated by the Doppler-mode processing circuitry 43. In addition, the acquiring function 453 acquires information about a type of Doppler mode that is obtained by converting an input operation received through the input interface 3 from the operator into an electric signal, the input operation indicating a selectable Doppler mode (selected), and output the electric signal.

The information about the type of Doppler mode is information for identifying a type of Doppler mode, such as tissue Doppler imaging-pulsed wave (TDI-PW), pulsed wave (PW), and continuous wave (CW), for example. Note that the information about the type of Doppler mode is not limited thereto.

Moreover, the acquiring function 453 acquires a position of the Doppler cursor (hereinafter, referred to as “cursor position information”) indicating a position where a blood flow velocity of the subject is measured from an ultrasonic image generated by the image generating function 452. When, for example, the acquired Doppler mode is PW, the acquiring function 453 acquires cursor position information corresponding to PW. The cursor position information is, for example, a cursor position of the left ventricular outflow tract (LVOT), a cursor position of the right ventricular outflow tract (RVOT), etc. Note that the cursor position information is not limited thereto.

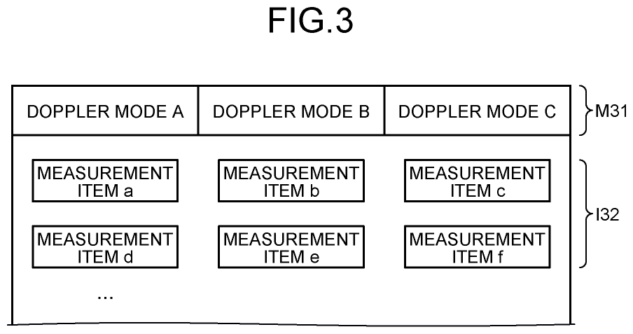

Explained below are measurement items for which the ultrasonic diagnosis apparatus 10 performs measurement. FIG. 3 is a schematic diagram illustrating exemplary operation buttons displayed on a touch command screen during image diagnosis according to the Doppler mode using the ultrasonic diagnosis apparatus 10 according to the embodiment. Doppler modes M31 include a plurality of modes including, for example, a Doppler mode A, a Doppler mode B, and a Doppler mode C. Types of the Doppler modes M31 corresponds to types of Doppler modes selectable by the operator.

Moreover, one or more measurement items 132 are provided for each type of Doppler mode. For example, measurement items executable in the Doppler mode A include a measurement item a, a measurement item b, a measurement item c, a measurement item d, a measurement item e, and a measurement item f.

There are displayed on the touch command screen some of the measurement items 132 that correspond to any Doppler mode M31 selected by the operator. The operator can make an instruction to execute measurement for a desired measurement item by manipulating an operation button. Note that the number of the Doppler modes M31 and the number of the measurement items 132 are not limited thereto.

As described above, for measurement based on the information about the type of Doppler mode, there exist many measurement items possible to become a target of measurement; therefore, the operator needs a time to decide which item is to be measured, and thus further improvement is required. To solve the problem, the processing circuitry 45 of the ultrasonic diagnosis apparatus 10 according to the present embodiment further includes the Doppler-mode determining function 454, the image recognition function 455, the measurement-item determining function 456, the measurement function 460, and the storage function 461.

The Doppler-mode determining function 454 is an example of a Doppler-mode determining unit. The image recognition function 455 is an example of an image recognition unit. The measurement-item determining function 456 is an example of a measurement-item determining unit. The measurement function 460 is an example of a measurement unit. The storage function 461 is an example of a storage unit.

Returning back to FIG. 1, the Doppler-mode determining function 454 determines a type of Doppler mode from the information about the type of Doppler mode acquired by the acquiring function 453. When, for example, the information about the type of Doppler mode acquired by the acquiring function 453 is information indicating TDI-PW, the Doppler-mode determining function 454 determines the information about the type of Doppler mode for which the operator performs measurement as TDI-PW.

When, for example, the information about the type of Doppler mode acquired by the acquiring function 453 is information indicating PW, the Doppler-mode determining function 454 determines the information about the type of Doppler mode for which the operator performs measurement as PW. When, for example, the information about the type of Doppler mode acquired by the acquiring function 453 is information indicating CW, the Doppler-mode determining function 454 determines the information about the type of Doppler mode for which the operator performs measurement as CW.

The image recognition function 455 recognizes an ultrasonic image generated by the image generating function 452. For example, the image recognition function 455 recognizes a cross-section of the circulatory system corresponding to a B-mode image generated by the image generating function 452 based on the trained model stored in the memory 44 that has been generated by using, as teacher data, B-mode images labelled with cross-sections of the circulatory system.

Examples of the cross-sections of the circulatory system include a long-axis view of the left ventricular apex, which is a cross-section for measurement of tricuspid regurgitation (TR), a short-axis view of the left ventricular aortic root in diastole or a short-axis view of the left ventricular aortic root in systole, which are cross-sections for measurement of the aortic valve (AV). Note that the cross-sections of the circulatory system are not limited thereto.

Moreover, for example, the image recognition function 455 recognizes a selectable measurement item corresponding to a Doppler image generated by the image generating function 452 based on the trained model stored in the memory 44 that has been generated by using, as teacher data, Doppler images labelled with selectable measurement items.

The selectable measurement item corresponding to the Doppler image is, for example, an E/A ratio where E is an early diastolic filling wave (E wave) and A is an atrial filling wave (A wave). Note that the selectable measurement item corresponding to the Doppler image is not limited thereto.

Note that, in the above-mentioned example, it has been explained the method in which the image recognition function 455 uses the trained models to recognize a B-mode image and a Doppler image generated by the image generating function 452. Embodiments, however, are not limited thereto. For example, it is allowable to recognize a B-mode image and a Doppler image from an ultrasonic image by means of other methods including a well-known pattern matching, an analysis based on an analytical feature point, etc.

The measurement-item determining function 456 determines at least one executable measurement item based on the information about the type of Doppler mode. When, for example, the Doppler-mode determining function 454 determines the information about the type of Doppler mode for which the operator performs measurement is TDI-PW, the measurement-item determining function 456 determines the executable measurement item as early diastolic wave of mitral annular motion e′.

When, for example, the Doppler-mode determining function 454 determines that the information about the type of Doppler mode for which the operator performs measurement is PW, the measurement-item determining function 456 determines that the at least one executable measurement item as one of the ratio of early diastolic wave to atrial systolic wave E/A, the LVOT, and the RVOT.

When, for example, the Doppler-mode determining function 454 determines that the information about the type of Doppler mode for which the operator performs measurement is CW, the measurement-item determining function 456 determines the at least one executable measurement item as either TR or AV.

The measurement-item determining function 456 includes a waveform determining function 457, a cursor-position determining function 458, and a cross-section determining function 459. The waveform determining function 457 is an example of a measurement-item determining unit and a waveform determining unit. The cursor-position determining function 458 is an example of the measurement-item determining unit and a cursor-position determining unit. The cross-section determining function 459 is an example of the measurement-item determining unit and a cross-section determining unit.

The waveform determining function 457 determines at least one executable measurement item based on a Doppler image generated from Doppler data. For example, the waveform determining function 457 determines whether the Doppler image is the ratio of early diastolic wave to atrial systolic wave E/A. More particularly, the waveform determining function 457 determines whether the selectable measurement item corresponding to the Doppler image recognized by the image recognition function 455 is the ratio of early diastolic wave to atrial systolic wave E/A.

The cursor-position determining function 458 determines whether the cursor position information is a cursor position in the LVOT. More particularly, the cursor-position determining function 458 determines whether the cursor position information acquired by the acquiring function 453 is a cursor position in the LVOT.

The cross-section determining function 459 determines at least one executable measurement item based on a B-mode image that is used for ultrasonic transmission/reception in a Doppler mode. For example, the cross-section determining function 459 determines whether the B-mode image is TR. More particularly, the cross-section determining function 459 determines whether a cross-section of the circulatory system corresponding to the B-mode image recognized by the image recognition function 455 is a cross-section for measurement of TR.

The measurement function 460 automatically executes at least one executable measurement item. More particularly, when the measurement-item determining function 456 determines that the information about the type of Doppler mode for which the operator performs measurement is TDI-PW, the measurement function 460 measures the early diastolic wave of mitral annular motion e′.

Then, when the measurement-item determining function 456 determines that the information about the type of Doppler mode for which the operator performs measurement is PW, the measurement function 460 measures an executable measurement item based on the result of determination by the measurement-item determining function 456.

More particularly, when the waveform determining function 457 determines that the Doppler image is the ratio of early diastolic wave to atrial systolic wave E/A, the measurement function 460 measures the ratio of early diastolic wave to atrial systolic wave E/A. Moreover, when the cursor-position determining function 458 determines that the cursor position information is a cursor position in the LVOT, the measurement function 460 measures the LVOT. Furthermore, when the cursor-position determining function 458 determines that the cursor position information is not a cursor position in the LVOT, the measurement function 460 measures the RVOT.

Then, when the measurement-item determining function 456 determines that the information about the type of Doppler mode for which the operator performs measurement is CW, the measurement function 460 measures an executable measurement item based on the result of determination by the measurement-item determining function 456.

More particularly, when the cross-section determining function 459 determines that the cross-section of the circulatory system corresponding to the B-mode image is a cross-section for measurement of TR, the measurement function 460 measures the TR. Moreover, when the cross-section determining function 459 determines that the cross-section of the circulatory system corresponding to the B-mode image is not a cross-section for measurement of TR, the measurement function 460 measures the AV.

The storage function 461 stores an ultrasonic image generated by the image generating function 452 in the memory 44. Moreover, the storage function 461 stores a Doppler mode and cursor position information acquired by the acquiring function 453 in the memory 44. Furthermore, the storage function 461 stores a result of determination by the Doppler-mode determining function 454 in the memory 44. The storage function 461 stores a result of image recognition by the image recognition function 455 in the memory 44. Moreover, the storage function 461 stores a result of determination by the measurement-item determining function 456 in the memory 44. Furthermore, the storage function 461 stores a result of measurement by the measurement function 460 in the memory 44.

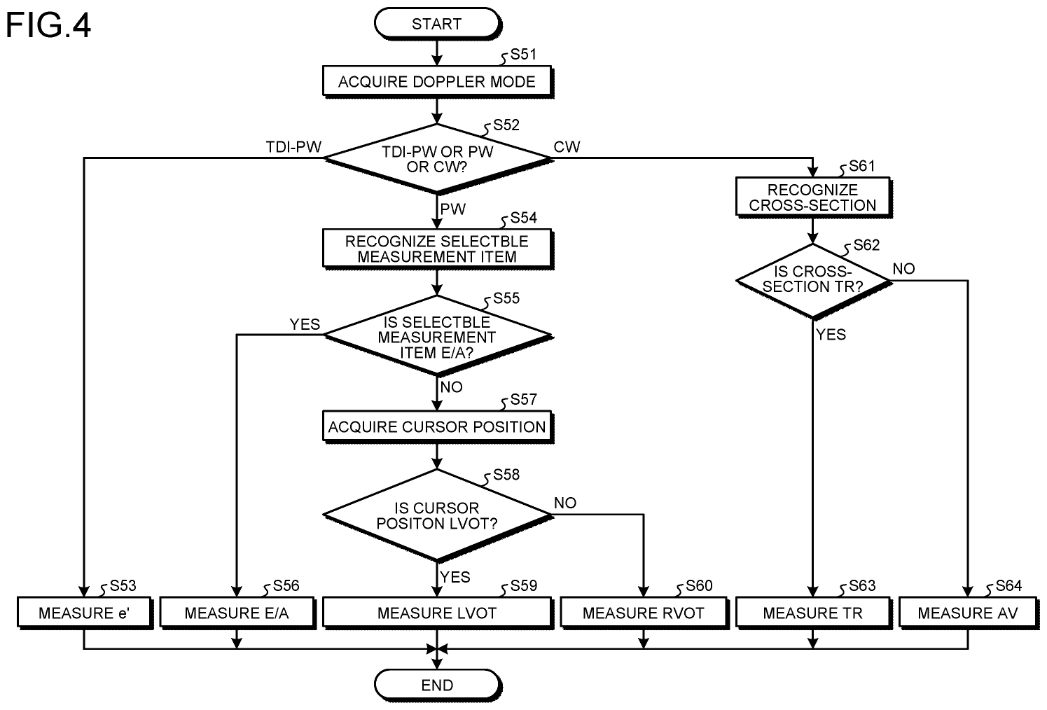

Explained below is processing procedure performed by the ultrasonic diagnosis apparatus 10 according to the embodiment with reference to FIG. 4. FIG. 4 is a flowchart for explaining the processing procedure performed by the ultrasonic diagnosis apparatus 10 according to the embodiment. Note that the present processing starts after the processing circuitry 45 collects ultrasonic images of the circulatory system and the operator presses a freeze button and then a measurement button.

The acquiring function 453 of the processing circuitry 45 acquires Doppler data acquired through ultrasonic transmission/reception in a Doppler mode and information about a type of Doppler mode (Step S51). Subsequently, the Doppler-mode determining function 454 of the processing circuitry 45 determines a Doppler mode from the information about the type of Doppler mode acquired by the acquiring function 453 (Step S52).

When the information about the type of Doppler mode acquired by the acquiring function 453 is TDI-PW, the Doppler-mode determining function 454 of the processing circuitry 45 goes to Step S53. Moreover, when the information about the type of Doppler mode acquired by the acquiring function 453 is PW, the Doppler-mode determining function 454 of the processing circuitry 45 goes to Step S54. Furthermore, when the information about the type of Doppler mode acquired by the acquiring function 453 is CW, the Doppler-mode determining function 454 of the processing circuitry 45 goes to Step S61.

When the Doppler-mode determining function 454 determines that the information about the type of Doppler mode acquired by the acquiring function 453 is TDI-PW, at Step S53, the measurement-item determining function 456 of the processing circuitry 45 determines the executable measurement item as the early diastolic wave of mitral annular motion e′. Then, when the measurement-item determining function 456 determines the executable measurement item as the early diastolic wave of mitral annular motion e′, the measurement function 460 of the processing circuitry 45 measures the early diastolic wave of mitral annular motion e′ (Step S53).

At Step S54, the image recognition function 455 of the processing circuitry 45 recognizes a selectable measurement item corresponding to the Doppler image generated by the image generating function 452 (Step S54). Subsequently, the waveform determining function 457 of the processing circuitry 45 determines whether the selectable measurement item corresponding to the Doppler image that is recognized by the image recognition function 455 is the ratio of early diastolic wave to atrial systolic wave E/A (Step S55).

When the waveform determining function 457 of the processing circuitry 45 determines that the selectable measurement item corresponding to the Doppler image that is recognized by the image recognition function 455 is not the ratio of early diastolic wave to atrial systolic wave E/A (No at Step S55), the process goes to Step S57. When the waveform determining function 457 of the processing circuitry 45 determines that the selectable measurement item corresponding to the Doppler image that is recognized by the image recognition function 455 is the ratio of early diastolic wave to atrial systolic wave E/A (Yes at Step S55), the process goes to Step S56.

When the waveform determining function 457 determines that the Doppler image is the ratio of early diastolic wave to atrial systolic wave E/A, at Step S56, the measurement function 460 of the processing circuitry 45 measures the ratio of early diastolic wave to atrial systolic wave E/A (Step S56).

At Step S57, the acquiring function 453 of the processing circuitry 45 acquires the cursor position information indicating the position where a blood flow velocity of the subject is measured (Step S57). Subsequently, the cursor-position determining function 458 of the processing circuitry 45 determines whether the cursor position information is a cursor position of the LVOT (Step S58).

When the cursor-position determining function 458 of the processing circuitry 45 determines that the cursor position information is not a cursor position of the LVOT (No at Step S58), the process goes to Step S60. When the cursor-position determining function 458 of the processing circuitry 45 determines that the cursor position information is a cursor position of the LVOT (Yes at Step S58), the process goes to Step S59.

When the cursor-position determining function 458 determines that the cursor position information is a cursor position of the LVOT, at Step S59, the measurement function 460 of the processing circuitry 45 measures the LVOT (Step S59).

When the cursor-position determining function 458 determines that the cursor position information is not a cursor position of the LVOT, at Step S60, the measurement function 460 of the processing circuitry 45 measures the RVOT (Step S60).

At Step S61, the image recognition function 455 of the processing circuitry 45 recognizes a cross-section of the circulatory system corresponding to the B-mode image generated by the image generating function 452 (Step S61). Subsequently, the cross-section determining function 459 of the processing circuitry 45 determines whether the cross-section of the circulatory system recognized by the image recognition function 455 that corresponds to the B-mode image is a cross-section for measurement of TR (Step S62).

When the cross-section determining function 459 of the processing circuitry 45 determines that the cross-section of the circulatory system recognized by the image recognition function 455 that corresponds to the B-mode image is not a cross-section for measurement of TR (No at Step S62), the process goes to Step S64. When the cross-section determining function 459 of the processing circuitry 45 determines that the cross-section of the circulatory system recognized by the image recognition function 455 that corresponds to the B-mode image is a cross-section for measurement of TR (Yes at Step S62), the process goes to Step S63.

When the cross-section determining function 459 determines that the cross-section of the circulatory system corresponding to the B-mode image is a cross-section for measurement of TR, at Step S63, the measurement function 460 of the processing circuitry 45 measures the TR (Step S63).

When the cross-section determining function 459 determines that the cross-section of the circulatory system corresponding to the B-mode image is not a cross-section for measurement of TR, at Step S64, the measurement function 460 of the processing circuitry 45 measures the AV (Step S64). The present process is completed when the process at Step S64 is completed. Note that the control function 451 of the processing circuitry 45 displays the result of measurement by the measurement function 460 of the processing circuitry 45 on the display 2.

As described above, the ultrasonic diagnosis apparatus 10 according to the embodiment acquires Doppler data that is obtained through ultrasonic transmission/reception in a Doppler mode and information about a type of Doppler mode, and it determines at least one executable measurement item for the acquired Doppler data based on the acquired information about the type of Doppler mode.

According to at least one embodiment as described above, when, for example, the operator presses a button to select a type of Doppler mode that is used for image diagnosis, the ultrasonic diagnosis apparatus 10 acquires Doppler data through ultrasonic transmission/reception according to the selected Doppler mode, and it also automatically measures at least one executable measurement item for the acquired Doppler data depending on the type of Doppler mode selected by the operator.

With this configuration, the operator can perform measurement for the circulatory system of the subject just by pressing a button to select a type of Doppler mode that is used for image diagnosis, without selecting an item to be measured from a plurality of measurement items. Accordingly, with the ultrasonic diagnosis apparatus 10 according to the present embodiment, it becomes possible to reduce operator's workloads for image diagnosis that uses an ultrasonic diagnosis apparatus and improve the workflow related to measurement.

### Modifications of Embodiments

With the above embodiment, it is explained an example in which the Doppler-mode determining function 454 determines the type of Doppler mode is any of TDI-PW, PW, and CW based on information about the Doppler mode for which the operator performs measurement, and the measurement-item determining function 456 of the processing circuitry 45 automatically measures at least one executable measurement item depending on the type of Doppler mode determined. In contrast, the control function 451 of the processing circuitry 45 according to a modification may display, on a touch command screen of the ultrasonic diagnosis apparatus 10, an operation button with which an instruction is made to execute an executable measurement item determined by the measurement-item determining function 456.

This excludes some operation buttons corresponding to measurement items unrelated to the Doppler mode currently used, which allows the operator to rapidly and easily select a measurement item required among the operation buttons corresponding to the executable measurement items only. Accordingly, with the ultrasonic diagnosis apparatus 10 according to the modification, it becomes possible to reduce operator's workloads and improve the workflow related to measurement.

Note that the term “processor” used in the above explanation means, for example, a central processing unit (CPU), a graphics processing unit (GPU), or circuits such as an application specific integrated circuit (ASIC), a programmable logic device (for example, a simple programmable logic device (SPLD), a complex programmable logic device (CPLD), and a field programmable gate array (FPGA)), etc. The processor implements a function by reading and executing a program that is stored in a memory.

Note that it is allowable to directly incorporate a program in circuitry of the processor, instead of storing the program in a memory. In this case, the processor implements the function by reading and executing the program that is incorporated in the circuitry. Note that each processor according to the present embodiment is not limited to form as a single circuit for each processor, and it is allowable to form a single processor by combining two or more independent circuits and implement its functionality.

Note that the respective components of the respective devices illustrated with reference to the explanation of the embodiments above are functionally conceptual ones, and the components are unnecessary to be provided physically as illustrated in the drawings. In other words, specific configuration for integration and distribution of devices is not limited to the configuration as illustrated, and all or part thereof can be functionally or physically distributed or integrated in any unit, depending on various types of load, usage status, etc. Moreover, all or part of the respective processing functions performed by the respective devices may be implemented by a CPU and a program that is analyzed and executed by the CPU or may be implemented as hardware by wired logic.

Moreover, the determination methods as described in the above embodiments can be implemented by a computer, such as a personal computer and a work station, executing a determination program prepared in advance. This determination program can be distributed via a network, such as the Internet. Moreover, this determination program can be stored in a non-transitory computer readable medium, for example, a hard disk, a flexible disk (FD), a CD-ROM, an MO, a DVD, a flash memory such as a USB memory and an SD card memory, and the program can be executed by a computer reading the program from the non-transitory computer readable medium.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. An ultrasonic diagnosis apparatus comprising a processor, the processor configured to:
acquire Doppler data that is obtained through ultrasonic transmission/reception in a Doppler mode and information about a type of the Doppler mode; and
determine at least one executable measurement item for the Doppler data based on the information about the type of the Doppler mode.

2. The ultrasonic diagnosis apparatus according to claim 1, wherein the processor automatically executes the at least one executable measurement item.

3. The ultrasonic diagnosis apparatus according to claim 1, wherein the processor displays, on a display device, an operation button with which an instruction is made to execute the at least one executable measurement item.

4. The ultrasonic diagnosis apparatus according to claim 2, wherein the processor determines the at least one executable measurement item based on a Doppler image generated from the Doppler data.

5. The ultrasonic diagnosis apparatus according to claim 2, wherein the processor determines the at least one executable measurement item based on cursor position information indicating a position of a Doppler cursor.

6. The ultrasonic diagnosis apparatus according to claim 2, wherein the processor determines the at least one executable measurement item based on a B-mode image that is used for the ultrasonic transmission/reception in the Doppler mode.

7. A medical image processing apparatus comprising a processor, the processor configured to:
acquire Doppler data that is obtained through ultrasonic transmission/reception in a Doppler mode and information about a type of the Doppler mode; and
determine at least one executable measurement item for the Doppler data based on the information about the type of the Doppler mode.

8. A non-transitory computer readable medium, the medium storing a program that, when executed by a computer, causes the computer to perform:
acquiring Doppler data that is obtained through ultrasonic transmission/reception in a Doppler mode and information about a type of the Doppler mode; and
determining at least one executable measurement item for the Doppler data based on the information about the type of the Doppler mode.
